(19) United States
(12) Patent Application Publication
(10) Pub. No.: US 2008/0145453 A1
(43) Pub. Date: Jun. 19, 2008

(54) Novel Dihydropyrimidine Derivatives And Their Use As Anti-Cancer Agents

(76) Inventors: Roman Lopez, Issy les Moulineaux (FR); Bernard Rousseau, Levallois-Perret (FR); Frank Kozielsky, Saint Egreve (FR); Dimitrios Skoufias, Grenoble (FR); Salvatore De Bonis, Grenoble (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/908,710

(22) PCT Filed: Mar. 14, 2006
(86) PCT No.: PCT/FR2006/000556

§ 371 Date: Nov. 6, 2007

(30) Foreign Application Priority Data
Mar. 25, 2005 (FR) 05 02518

(51) Int. Cl. A61K 33/24; C07D 239/10; A61K 31/505; A61P 35/00; A61P 31/00; A61P 9/00; A61P 25/00; A61P 37/00; A61K 31/4985; A61K 31/704

(52) U.S. Cl. 424/649; 544/318; 514/274; 514/34; 514/252.1

(57) ABSTRACT

The invention concerns molecules of formula (I), drugs containing same and their use as anti-cancer agents.

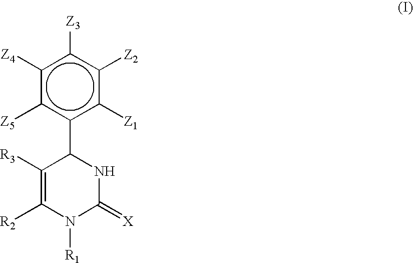

The present invention relates to novel molecules derived from dihydropyrimidine, to the medicaments containing them and to their use as anti-cancer agents.

One of the strategies developed for several years for the treatment of cancers consists in targeting proteins which play an important role in cell division. Eg5, which belongs to the kinesin motor protein family, involved in cell proliferation, is one of these proteins. Thus, a molecule which inhibits Eg5 is liable to be active in anti-cancer therapy.

This is the case of Monastrol, which is a dihydropyrimidine derivative that inhibits the kinesin Eg5 (Mayer T. U. et al, Science, 296 (5441), 971-974, 1999; Kapoor, J. Cell. Biol. 150(5), 975-980, 2000). However, the antimitotic effect of Monastrol is weak (IC₅₀>14 μm) and does not make it possible to envision its use as an anti-cancer treatment.

International applications WO 02/079149 and WO 02/079169 describe Monastrol-derived compounds of cyanodihydropyrimidine type. However, the most active among these compounds have an antimitotic activity that is only slightly better than that of Monastrol (IC₅₀≦10 μm)

Furthermore, even when the antimitotic activity of a compound is insufficient in itself for treating a cancer, a compound which has an antimitotic activity, in particular an Eg5-inhibiting activity, can be used in a polytherapy involving several distinct targets.

Consequently, there remains the need for compounds which have an Eg5-inhibiting activity of a level sufficient to make it possible to envision their use in an anti-cancer therapy, alone or in combination with other active ingredients.

The compounds of the present invention possess an Eg5-protein-modulating activity, in particular an Eg5-protein-inhibiting activity. They have an antimitotic effect and, in this respect, can be used for the preparation of a medicament for use in the prevention or treatment of a cancer.

The invention relates more particularly to the molecules corresponding to formula (I):

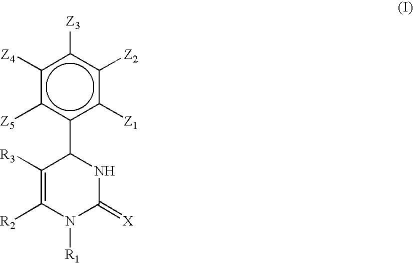

in which
X represents an atom chosen from: O and S;
R₁ represents a group chosen from: a hydrogen atom, a C₁-C₆ alkyl group, a C₂-C₆ alkenyl group, a C₁-C₆ haloalkyl group, a C₆-C₁₂ aralkyl group and a phenyl group optionally substituted with one or more groups chosen from: a halogen, a C₁-C₃ alkyl, a C₁-C₃ haloalkyl, a hydroxyl group and a C₁-C₃ alkoxy group;
R₂ represents a group chosen from: a C₁-C₆ alkyl group, a C₂-C₆ alkenyl group, a C₁-C₆ haloalkyl group, a C₆-C₁₂ aralkyl group and a phenyl group optionally substituted with one or more groups chosen from: a halogen, a C₁-C₃ alkyl, a C₁-C₃ haloalkyl, a hydroxyl group and a C₁-C₃ alkoxy group;
R₃ represents a group chosen from:

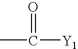

and Y₁ represents a group chosen from: a group -Q₁, a group —OQ₁, a group —NHQ₁ and a group —NQ₁Q₂;

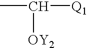

and Y₂ represents a group chosen from: H, a group -Q₂ and a group —OCOQ₂;

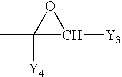

and one of Y₃ and Y₄ represents H, while the other represents a group -Q₁;

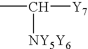

Y₇ represents a group chosen from: H and a group -Q₁;
and either
Y₅ represents a group chosen from H and a group -Q₂, and Y₆ is chosen from: a hydrogen atom and a group chosen from: -Q₃, —COQ₃ and —CO₂Q₃; or
Y₅ represents H, and Y₆ is chosen from a group —SO₂Q₃ and a group —CONHQ₃;
—CH₂Y₈ and Y₈ is chosen from one of the following groups: -Q₁, —OQ₁, —OCONHQ₁, —OCONQ₁Q₂, —NHQ₁, —NQ₁Q₂, —NHCOQ₁, —NQ₁COQ₂, —NQ₁CO₂Q₂, —NHCO₂Q₁, —NHSO₂Q₁ and —NHCONHQ₁;
and Q₁, Q₂ and Q₃, which may be identical or different, represent a group chosen from: a C₁-C₆ alkyl group, a C₂-C₆ alkenyl group, a C₁-C₆ haloalkyl group, a C₆-C₁₂ aralkyl group, a heteroaryl group containing up to 12 carbon atoms and from 1 to 3 heteroatoms, and a phenyl optionally substituted with one or more groups chosen from: a halogen, a C₁-C₆ alkyl, a C₁-C₆ haloalkyl, a hydroxyl group, a C₁-C₆ alkoxy group and a C₆-C₉ aralkyl group;
z₁, Z₂, Z₃, Z₄ and Z₅, which may be identical or different, being chosen from: a hydrogen atom, a halogen, a hydroxyl group, a C₁-C₆ alkyl group, a C₁-C₆ haloalkyl group, a C₁-C₆ alkoxy group and a C₁-C₁₂ acyloxy group;
enantiomers thereof, diastereoisomers thereof and pharmaceutically acceptable salts thereof,
it being understood that:
when R₁=H, X=S, R₂=CH₃, Z₁=Z₂=Z₃=Z₅=H and Z₄=OH,
R₃ is different than

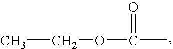

when R₁=H or R₁=CH₃, R₂=CH₃, R₃=—CO—CH₃ and Z₁=Z₂=Z₄=Z₅=H, then Z₃≠H,
when R₁=H,

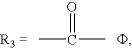

and Z₁=Z₂=Z₃=Z₄=Z₅=H, then R₂≠—CF₂—CF₂H, CH₃.

The term “C₁-C₆ alkyl” denotes a linear, branched or cyclic hydrocarbon-based radical containing from 1 to 6 carbon atoms; mention may, for example, be made of methyl, propyl, n-butyl, isopropyl, isobutyl, tert-butyl, n-pentyl, hexyl, isohexyl, 1-ethylpropyl, etc.

The term “C₂-C₆ alkenyl” denotes a linear, branched or cyclic hydrocarbon-based radical containing at least one double bond and 2 to 6 carbon atoms. Mention may, for example, be made of propen-2-yl.

The term “aralkyl” denotes a linear, branched or cyclic alkyl radical substituted with an aryl group, such as, for example, a pyridine or a phenyl, itself optionally substituted with one or more groups chosen from: a halogen, a C₁-C₃ alkyl, a C₁-C₃ haloalkyl, a hydroxyl group and a C₁-C₃ alkoxy group.

The term “C₁-C₆ haloalkyl” denotes a linear, branched or cyclic C₁-C₆ alkyl radical substituted with at least one halogen atom, such as F, Br, I or Cl. Mention may, for example, be made of: —CF₃ and —CH₂—CH₂Cl.

The halogen atom can be chosen from the following list: F, Cl, Br and I.

The C₁-C₆ alkoxy group denotes a radical —OW in which W represents a C₁-C₆ alkyl. Mention may, for example, be made of a methoxy, ethoxy or isopropoxy radical.

The C₁-C₁₂ acyloxy group denotes a radical —O(CO)W′ in which W′ represents a C₁-C₁₂ alkyl. Mention may, for example, be made of an acetyl radical.

In the heteroaryl group containing up to 12 carbon atoms and from 1 to 3 heteroatoms, the heteroatoms can be preferably chosen from S, N or O. As examples, mention may be made of pyridine, quinoline and heteronaphthyl groups.

Preferably, R₂ is selected from C₁-C₆ alkyl and C₁-C₆ haloalkyl groups. For example, R₂ can be chosen from: —CH₃, —CH₂—CH₃, —CH(CH₃)₂, —CH₂—CH(CH₃)₂ and —CF₃.

Also preferably, at most just one of the groups Z₁, Z₂, Z₃, Z₄ and Z₅ is different than H, i.e. (Z₁, Z₂, Z₃, Z₄, Z₅)=(H, H, H, H, Y) and Y represents a group chosen from a hydrogen atom, a hydroxyl group, a halogen atom, a C₁-C₆ alkyl group, a C₁-C₆ haloalkyl group, a C₁-C₆ alkoxy group and a C₁-C₁₂ acyloxy group. The group Y can be any of Z₁, Z₂, Z₃, Z₄ or Z₅, i.e. it can be in the ortho-, meta- or para-position on the aromatic ring.

Advantageously, Y is chosen from: —OH, H, Cl, F, Br and —OCH₃.

More advantageously, (Z₁, Z₃, Z₄, Z5)=(H, H, H, H) and Z₂ represents a group chosen from a hydroxyl group and a C₁-C₆ alkoxy group.

Preferably, in formula (I), one of the following conditions is met:
Z₁=Z₂=Z₄=Z₅ =H and Z₃=—OH;
Z₁=Z₂=Z₄=Z₅ =H and Z₃=—OCH₃;
Z₁=Z₃=Z₄=Z₅ =H and Z₂=—OH;
Z₁=Z₃=Z₄=Z₅ =H and Z₂=—OCH₃;
Z₁=Z₂=Z₃=Z₄ =Z₅=H;
Z₁=Z₂=Z₃=Z₄ =H and Z₅ is chosen from F, Cl and Br;
Z₁=Z₂=Z₃=Z₅ =H and Z₄ is chosen from F, Cl and Br.

According to a first preferred variant, the invention relates to the molecules of formula (I) in which R₁ represents a group chosen from: a C₁-C₆ alkyl group, a C₂-C₆ alkenyl group, a C₁-C₆ haloalkyl group and a phenyl group optionally substituted with one or more groups chosen from: a halogen, a C₁-C₃ alkyl, a C₁-C₃ haloalkyl, a hydroxyl group and a C₁-C₃ alkoxy group.

Preferably, R₁ is selected from C₁-C₃ alkyls and alkenyls. Advantageously, R₁ is chosen from the groups —CH₃ and —CH₂—CH═CH₂.

According to this variant, R₃ is advantageously chosen from the groups

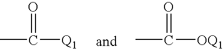

in which Q₁ is as defined above. Among the latter, the preferred groups R₃ are:
the groups

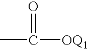

in which Q₁ is chosen from C₁-C₃ alkyls, optionally substituted phenyls and C₆-C₉ aralkyls; in particular, mention may be made of —CH₂—CH₃ and —CH₂-Φ, Φ representing the phenyl ring;
the groups

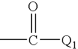

in which Y₁=Q₁ is chosen from C₁-C₃ alkyls and optionally substituted phenyls; in particular, mention made be made of methyl, meta-fluorophenyl, meta-iodophenyl, meta-chlorophenyl and meta-methoxyphenyl groups.

According to a second preferred variant of the invention, R₃ is chosen from the groups corresponding to the formula

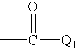

also denoted

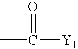

in which Q₁=Y₁ is as defined above. Advantageously, according to this variant, the group Q₁=Y₁ is chosen from C₁-C₆ alkyl groups, even more preferably C₁-C₃ alkyl groups, or from optionally substituted phenyl groups. In the latter case, the phenyl group is advantageously substituted with one or more substituents chosen from: —OCH₃, —F, —Cl and —Br.

Preferably, Q₁=Y₁ is chosen from methyl, phenyl, meta-fluorophenyl, meta-iodophenyl, meta-chlorophenyl and meta-methoxyphenyl.

According to this variant, R₁ is preferably chosen from the following list: H, —CH₃ and —CH₂—CH═CH₂.

Advantageously, the compounds of the invention are chosen from those numbered 1 to 34, and the formula of which is given below:

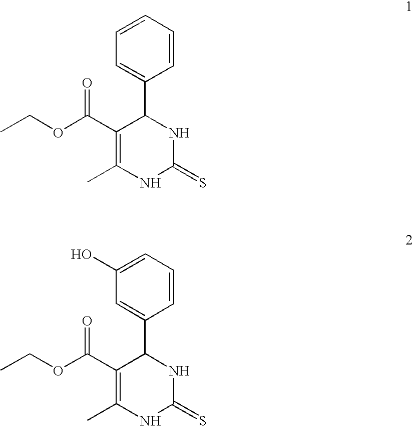

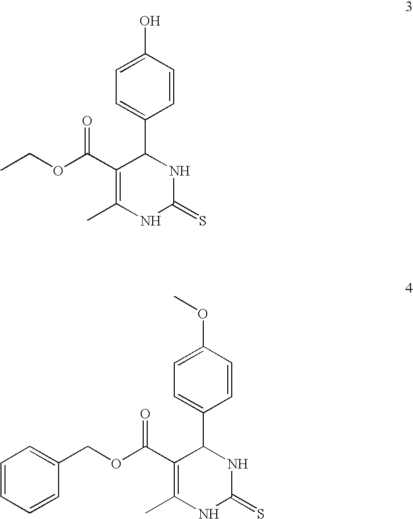

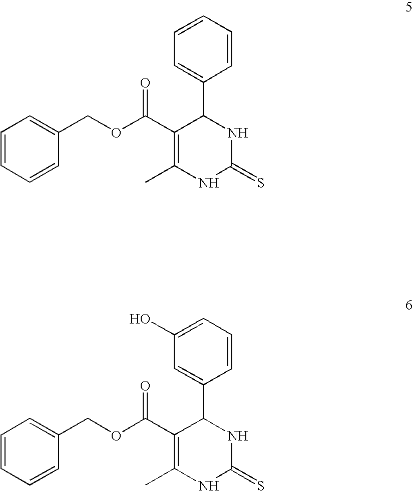

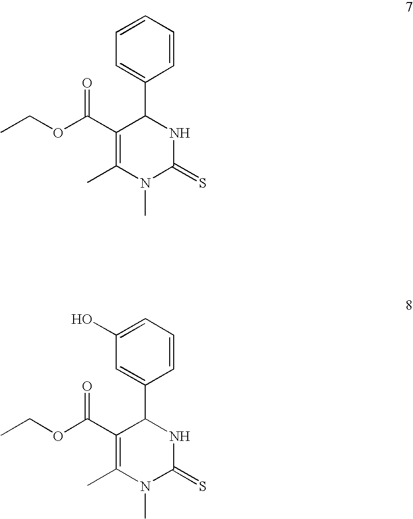

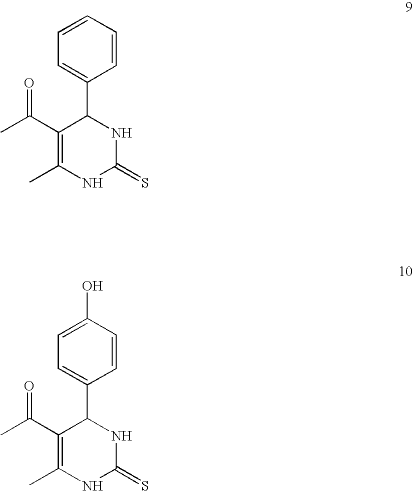

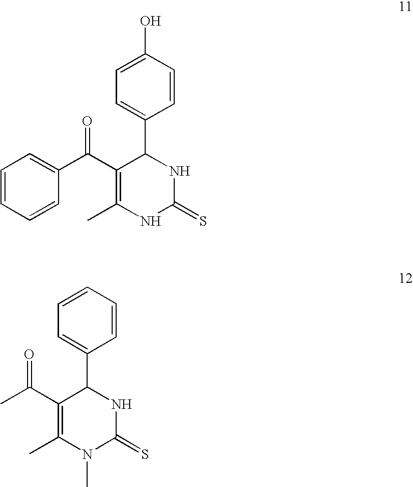

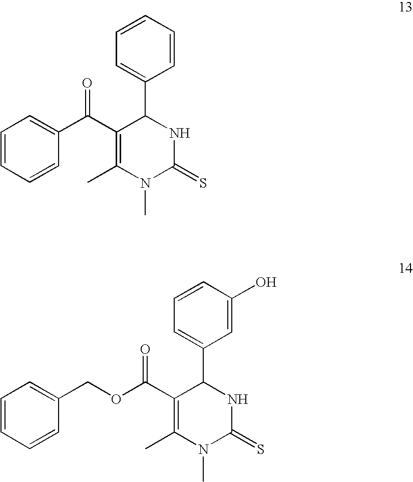

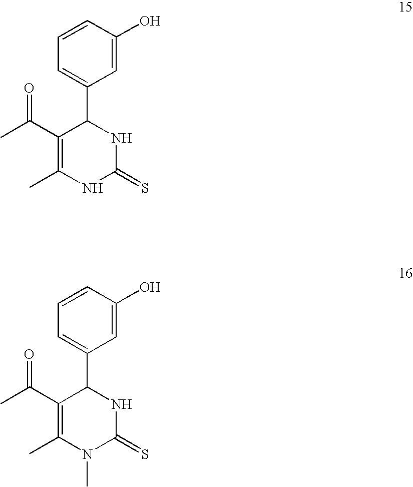

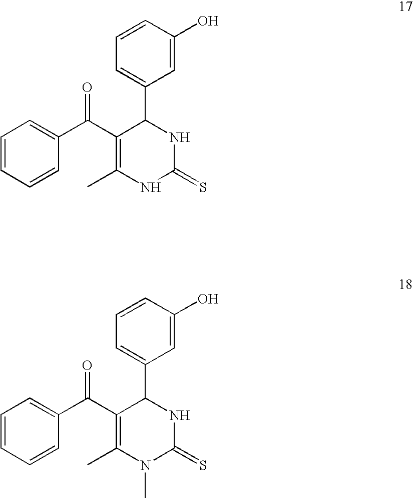

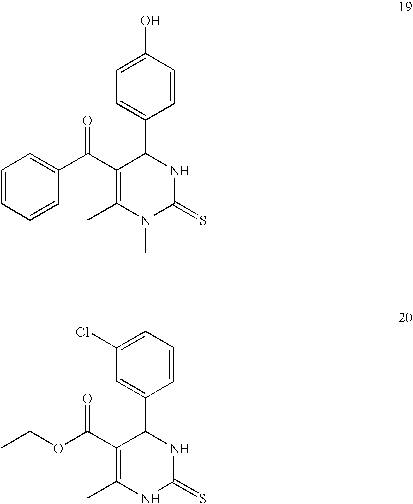

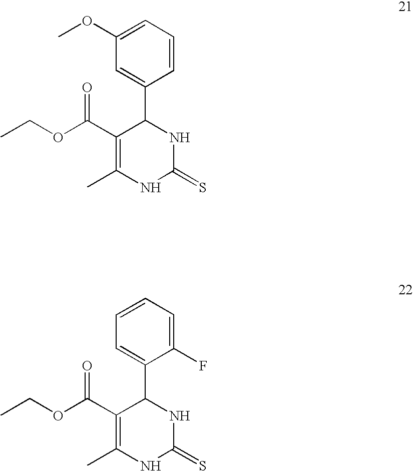

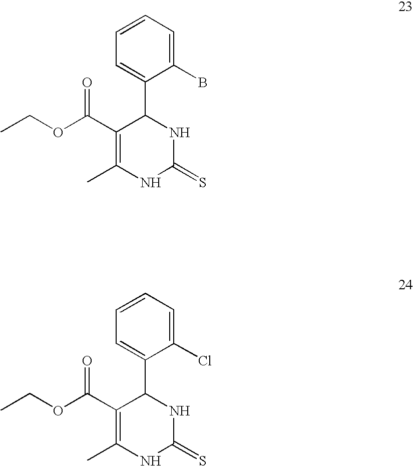

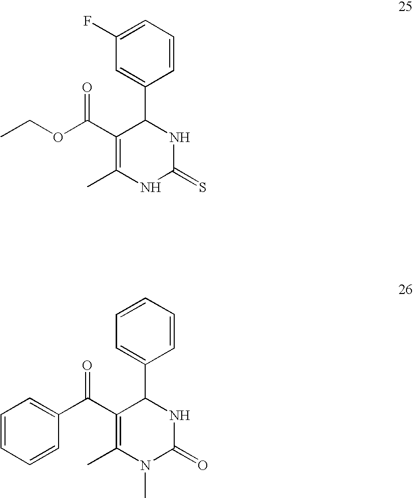

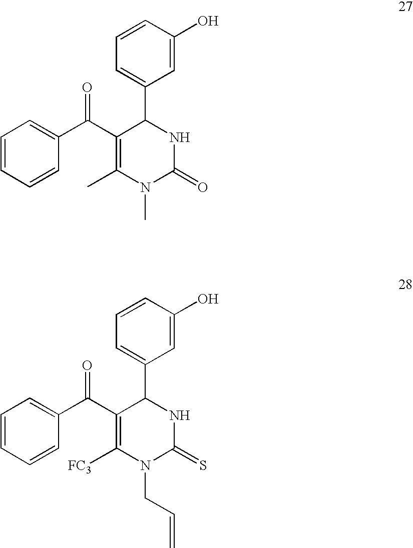

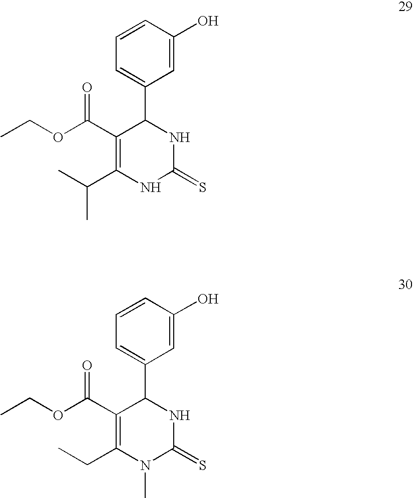

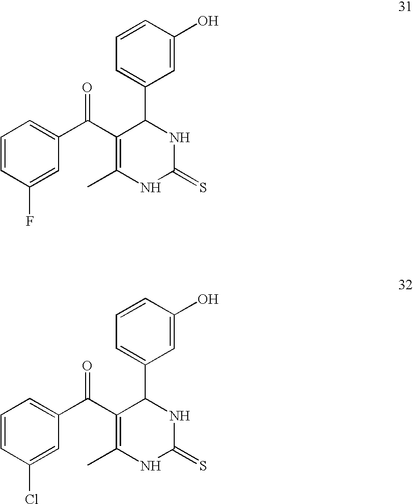

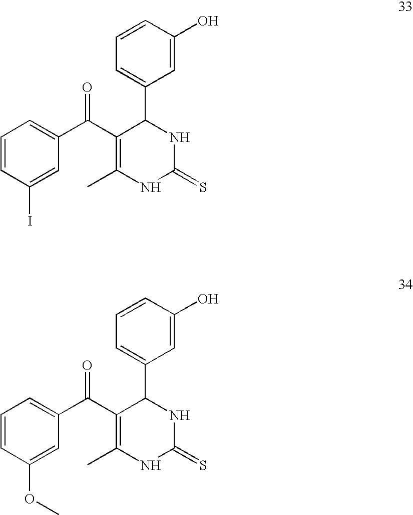

The compounds of the invention can be prepared according to a method of synthesis disclosed in scheme 1 below:

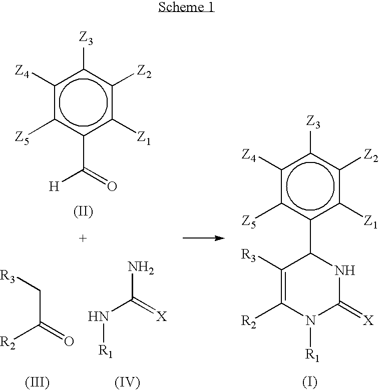

The aldehyde (II), the ketone (III) and the thiourea (IV) are reacted together so as to give the compound (I) according to the present invention.

Depending on the nature of the substituents R₁, R₂, R₃, Z₁, Z₂, Z₃, Z₄ and Z₅ in compound (I), it may be envisaged to carry out the reaction on compounds of formula (II), (III) and (IV) in which these functions are protected, the reaction for formation of the 2-thioxo-1H,3H-pyrimidine ring then being followed by the deprotection of the functions which were protected. These protection/deprotection reactions are well known to those skilled in the art. Reference may, for example, be made to Ronald R. C. et al., J. Org Chem. 1982, 47, 2541.

According to a first variant of the process of the invention, the three reactants (II), (III) and (IV) are reacted in the absence of solvent. The aldehyde (II) and the ketone (III) are in an amount substantially equivalent in terms of number of moles, whereas the thiourea (IV) is present in excess. Preferably, the thiourea (IV) is used in an amount of between 1 and 2 times the amount of the aldehyde (II) or of the ketone (III). Preferably, the mixture is heated to a temperature ranging from 80 to 120° C. for several hours. A catalyst can optionally be used. This catalyst can, for example, be chosen from Yb(OTF)₃, Sc(OTf)₃, La(OTf)₃, YbCl₃, InCl₃ and concentrated HCl.

The resulting product is purified by methods well known to those skilled in the art: crystallization, precipitation, silica gel chromatography, extraction, high performance liquid chromatography.

According to a variant of the invention, the reaction described in scheme 1 can be carried out in the solid phase, compound (II) being attached to a solid resin via one of its functionalities Z₁, Z₂, Z₃, Z₄ or Z₅. Such a variant is illustrated in scheme 2 in the case where Z₄=OH:

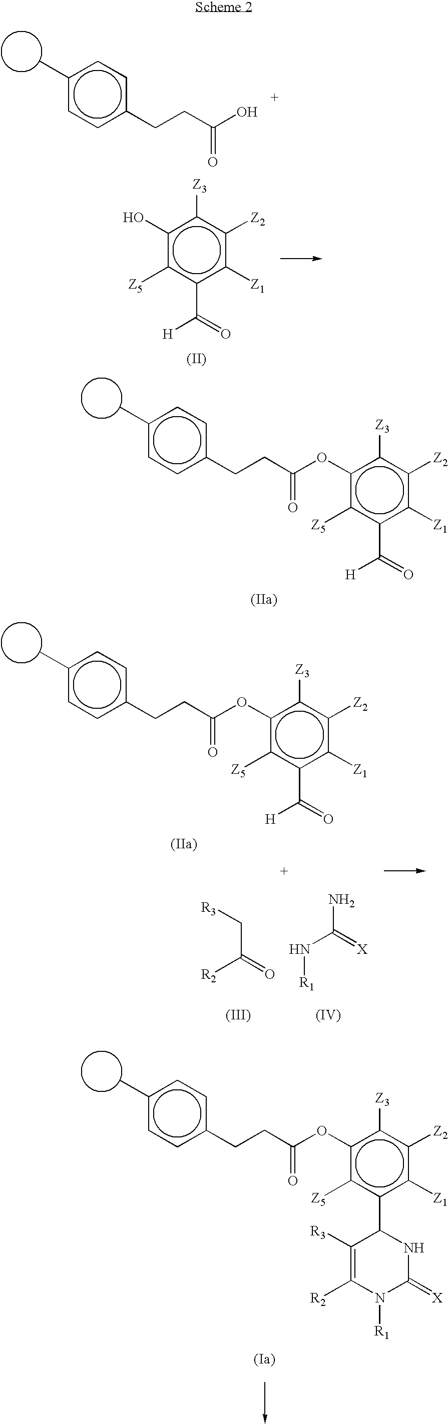

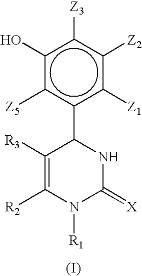

According to this variant of the invention, compound (II) is grafted onto a resin comprising a carboxylic acid functionality by means of an esterification reaction, so as to give the functionalized resin (IIa). This resin (IIa) is placed in the presence of the ketone (III) and the thiourea (IV) so as to give the grafted resin (Ia) from which compound (I) can be detached by simple hydrolysis.

In a known manner, the grafting of (II) onto the acid resin is carried out using a coupling agent such as DCC. Preferably, the reaction of the grafted resin (IIa) with the ketone (III) and the thiourea (IV) is carried out as disclosed above without solvent, at a temperature of between 80 and 120° C. in the presence of a catalyst and/or by applying a microwave treatment. When a catalyst is used, it is possible, for example, to choose said catalyst from Yb(OTF)₃, Sc(OTf)₃, La(OTf)₃, YbCl₃, InCl₃ and concentrated HCl.

A subject of the invention is also a medicament comprising a compound of formula (I) as described above, in a pharmaceutically acceptable carrier. The compounds of the invention and the medicaments containing them are more particularly for use in the prevention and/or treatment of a proliferative disease such as a cancer. These compounds and these medicaments have the property of modulating the activity of the motor protein Eg5 and/or of inducing apoptosis. They have an antimitotic activity and, consequently, they can be used for the prevention and/or treatment of various pathologies of proliferative type, such as cancers, autoimmune diseases, viral diseases, fungal diseases, neurodegenerative diseases and cardiovascular diseases.

The molecules and the medicaments of the invention are particularly useful for the prevention and/or treatment of cancers, in particular: lung cancer, breast cancer, pancreatic cancer, stomach cancer, ovarian cancer, esophageal cancer, thyroid cancer, prostate cancer, melanomas, lymphomas, sarcomas, carcinomas, and nervous system tumors.

Among the proliferative diseases, mention may be made of adenomatous polyposis and atherosclerosis.

Among the viral diseases, mentioned may be made of HIV infection and herpes. Among the autoimmune diseases, mention may be made of inflammatory bowel diseases, psoriasis and autoimmune diabetes. Among the neurodegenerative diseases, mention may be made of Alzheimer's disease and Parkinson's disease.

A subject of the invention is also the use of a compound of formula (I) as described above, for the preparation of a medicament for use in the prevention and/or treatment of a proliferative disease such as a cancer. In particular, the molecules of the invention are also useful for blocking the development of a cancer, in particular by blocking the progression of malignant cells or by inhibiting the development of the tumor.

The compounds and the medicaments of the invention can be used alone or in combination with another molecule or medicament known as an anti-cancer treatment, such as, for example, doxorubicin, paclitaxel, etoposide, cisplatin, tamoxifen, methotrexate or 5-fluorouracil.

The amount of molecule of formula (I) to be administered to humans, or optionally to animals, depends on the activity specific to this molecule, which activity can be measured by means which will be disclosed in the examples. It also depends on the degree of seriousness of the pathology to be treated, and on the age and the weight of the individual.

### EXAMPLES

### I—Synthesus of the Molecules

1—General Data:

The reactants used are commercial products. The ¹H and ¹³C NMR spectra were generated on Brucker® AC300 (300 MHz) and AVANCE400 (400 MHz) machines. The chemical shifts are given in ppm (δ), tetramethylsilane being used as a reference. The HPLC-mass analyses were carried out on a Waters® apparatus with UV detection and light scattering (DEDL).

2—Method of Synthesis No. 1:

In a Wheaton glass tube closed with a stopper, a mixture of aldehyde (1 mmol), of β-keto ester or of β-diketone (1 mmol) and thiourea (1.5 mmol) are heated at 110° C. without stirring for 3.5 hours. In certain cases, a catalyst, ytterbium triflate, is used in an amount of 5 mol %. The product obtained is isolated by one of the following methods:

a—The mixture is left to cool to ambient temperature. Ether (4 ml) is added thereto. The resulting solid is filtered, washed with ether (2×1 ml) and then with water (2×1 ml) and dried.

If no product precipitates after the addition of ether, the latter is evaporated off and the residue is purified;

b—by filtration on a microcolumn of silica (1 g of silica) with a solvent gradient of hexane up to a 7/3 hexane/EtOAc mixture; or
c—by dissolution in DMSO and purification by HPLC.

3—Method of Synthesis No. 2 (in Solid Phase):

Example with Z₄=OH, Z₁=Z₂=Z₃=Z₅=H

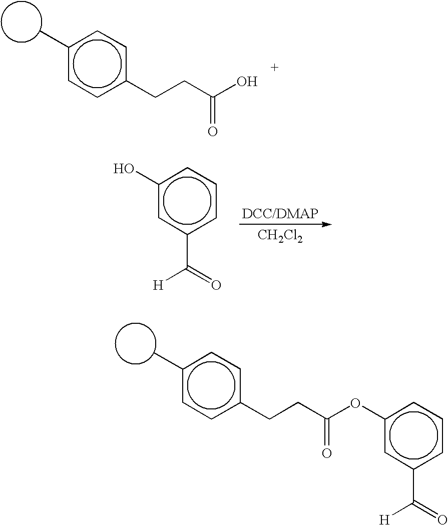

In a Wheaton glass tube closed with a stopper, the resin (2.22 mmol) is swollen in CH₂Cl₂ (20 ml) and, after 10 min, a solution of DCC (4.44 mmol) and DMAP (catalytic) in CH₂Cl₂ (3 ml) is added. After 10 min at ambient temperature, a solution of 3-hydroxybenzaldehyde (4.44 mmol) in THF (7 ml) is added and the mixture is stirred for 48 hours. The resin is filtered off, washed successively with 2×20 ml of CH₂Cl₂, MeOH, THF, MeOH and THF, and vacuum-dried to give a pale yellow resin.

IR (KBr) 1750-1650 Cm⁻¹ (broad band C═O).

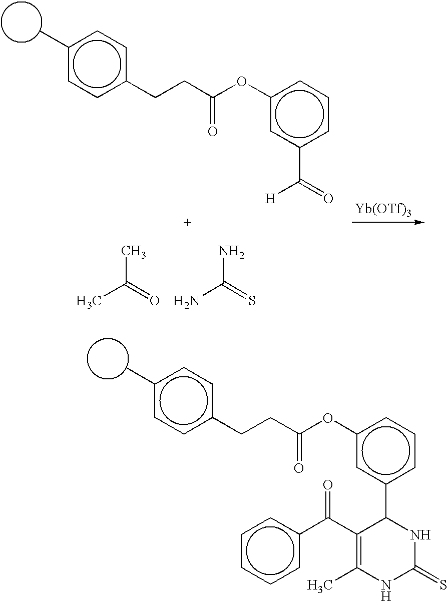

The β-diketone is introduced into a Wheaton glass tube closed with a stopper, followed by the addition of the powdered thiourea (0.3 mmol) and of a catalytic amount of ytterbium triflate. The resin (0.1 mmol) is then added. The amount of diketone provided is such that the resin is covered; the mixture is heated at 110° C. for 4 hours with vigorous stirring. Methanol is added and the resin is filtered off, washed successively with 2×20 ml of CH₂Cl₂, MeOH, THF, 1:1 THF/H₂O, MeOH and THF, and vacuum-dried to give a brown resin.

IR (KBr): 1400-1000 cm⁻¹ (broad band NC═SN).

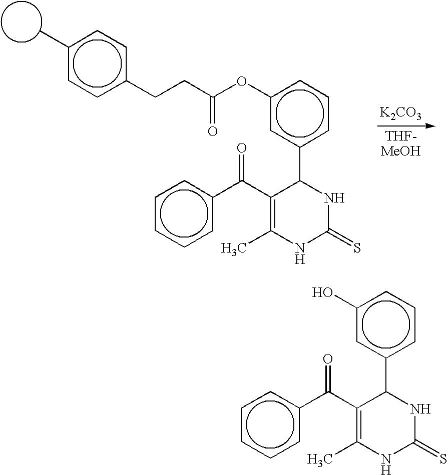

The resin is swollen in dry THF (4 ml) and a suspension of K₂CO₃ (6 eq) in methanol (2 ml) is added. The mixture is stirred at ambient temperature for several hours and the resin is filtered off and washed twice with methanol. The crude material is then adsorbed onto the silica and purified by filtration on a silica microcolumn (1 g of silica) with a solvent gradient of a 7/3 hexane/Et₂O mixture to Et₂O (purification method d).

All the products were analyzed by HPLC-MS and showed a purity greater than 99%.

Compound 7

¹H NMR (DMSO-cfe): δ 9.84 (d, J=4.3 Hz, 1H), 7.40-7.15 (m, 5H), 5.20 (d, J=4.3 Hz, 1H), 4.10 (q, J=6.7 Hz, 2H), 3.50 (s, 3H), 3.40 (s, 3H), 1.15 (t, J=6.7 Hz, 3H).

Compound 10

¹H NMR (DMSO-de): δ 10.19 (s, 1H), 9.65 (s, 1H), 9.43 (s, 1H, Ar—OH), 7.02 (d, J=7.8 Hz, 2H), 6.71 (d, J=7.8 Hz, 2H), 5.17 (d, J=3.7 Hz, 1H), 2.32 (s, 3H), 2.09 (s, 3H).

### II-Biological Activity

1—Measurement of the Amount of ATPase:

All the assays were carried out at ambient temperature using the TECAN 96-well Sunrise photometer with a final volume of 200-250 μl per well. The ATPase contents were measured using the pyruvate kinase/lactate dehydrogenase protocol in an A25A buffer (25 mM potassium ACES, pH 6.9, 2 mM magnesium acetate, 2 mM potassium EGTA, 0.1 mM potassium EDTA, 1 mM mercaptoethanol) . In the absence of MT (microtubules) stabilized with paclitaxel, 300 nM of Eg52-386 were used for the test and, in the absence of MT, the basal ATPase activity was measured using 1-4 μM of Eg5 (2-386) for the above test or the malachite green test. For optimal solubility of the inhibitor, the assays were carried out in the presence of up to 2.2% of DMSO. A control assay at this concentration of DMSO showed no effect on the MT-activated ATPase activity. The data were analyzed using a Kaleidagraph 3.0 (Synergy Software) and Microsoft Excel, so as to obtain the IC50 values.

2—Determination of the IC50 Values by In Vitro Inhibition of the ATPase Activity:

The IC50 values for the in vitro inhibition of the ATPase activity of the kinesin motor proteins are determined according to the method described in S. DeBonis et al. (2003) Biochemistry 42, 338-349. Monastrol was used as a positive control. When necessary, the concentrations of inhibitor were adjusted according to the initial IC50. Each concentration of inhibitor was measured one to three times and averaged data are indicated with the margins of error±the standard deviation.

Cell culture, immunofluorescence microscopy: HeLa cells were cultured on a Dulbecco's Modified Eagle medium (GIBCO, BRL) supplemented with 10% of bovine calf serum (Hyclone), and maintained in a humid incubator at 37° C. under 5% CO₂. The cells were left to adhere for at least 36 h on glass coated with poly-D-lysine in 24-well plates before adding the test compounds. After incubation with the test compounds for 8 h, the cells were fixed with 1% para-formaldehyde-PBS at 37° C. for 3 min, followed by incubation in 100% methanol at −20° C. for 5 min, and washed with PBS for 5 min. After two further washes for 5 minutes, the fixed cells were incubated with anti-alpha-tubulin YL1/2 for 1 hour and then with an FITC-conjugated goat anti-rat secondary antibody (Jackson ImmunoResearch Laboratories, West Grove, Pa., USA) for 30 min, and revealed with propidium iodide. The images were obtained with an MRC-600 scanning laser confocal device (BioRAD Laboratories) coupled to a Nikon Optiphot microscope.

Purification of human Eg5: The purification of human Eg5 (monomeric construct Eg52-386) has already been described (DeBonis, S., et al., (2003). Interaction of the mitotic inhibitor monastrol with human kinesin Eg5. Biochemistry 42, 338-349).

The methods of synthesis and the results of the in vitro biological assays are disclosed in table 1. The IC50 values and the percentage inhibitions are given in the table below.

TABLE 1

| | | Synthesis | Isolation | | | % | IC50 |
|---|---|---|---|---|---|---|---|
| Compound | Catalysis | protocol | method | Purity | Yield | inhibition | (μm) |
| Compound 1 | no | 1 | a | >99% | 53 | 80% | 9.5 |
| Compound 2 | yes | 1 | b | >99% | 38 | ≧90% | 16.0 |
| Compound 3 | no | 1 | c | >99% | — | 20% | 212.0 |
| Compound 4 | no | 1 | b | >99% | 32 | 40% | 6.6 |
| Compound 5 | no | 1 | b | >99% | 17 | 40% | 11.8 |
| Compound 6 | no | 1 | b | >99% | 17 | ≧90% | 10.30 |
| Compound 7 | no | 1 | c | >99% | — | 60% | 21.0 |
| Compound 8 | no | 1 | c | >99% | — | ≧90% | 6.30 |
| Compound 9 | no | 1 | a | >99% | 78 | ≧90% | 24.0 |
| Compound 10 | no | 1 | a | >99% | 73 | 80% | 56.0 |
| Compound 11 | no | 1 | a | >99% | 54 | ≧90% | 14.70 |
| Compound 12 | yes | 1 | c | >99% | — | 40% | 80.0 |
| Compound 13 | yes | 1 | c | >99% | — | ≧90% | 1.10 |
| Compound 14 | yes | 1 | c | >99% | — | 48% | 9.8 |
| Compound 15 | yes | 1 | c | >99% | — | ≧90% | 0.51 |
| Compound 16 | yes | 1 | c | >99% | — | 40% | 133 |
| Compound 17 | yes | 1 | c | >99% | — | ≧90% | 0.25 |
| Compound 18 | yes | 1 | c | >99% | — | ≧90% | 0.15 |
| Compound 19 | yes | 1 | c | >99% | — | 87% | 24.0 |
| Compound 20 | yes | 1 | a | >99% | 27 | 60% | 10.0 |
| Compound 21 | yes | 1 | a | >99% | 20 | 20% | 57.0 |
| Compound 22 | yes | 1 | c | >99% | — | 60% | 20.0 |
| Compound 23 | yes | 1 | c | >99% | — | 60% | 19.0 |
| Compound 24 | yes | 1 | c | >99% | — | 60% | 32.0 |
| Compound 25 | yes | 1 | a | >99% | 57 | 60% | 14.0 |
| Compound 26 | yes | 1 | c | >99% | — | 84% | 5.0 |
| Compound 27 | yes | 1 | c | >99% | — | 85% | 0.8 |
| Compound 28 | yes | 1 | c | >99% | — | 70% | 17.0 |
| Compound 29 | yes | 1 | c | >99% | — | 80% | 8.5 |
| Compound 30 | yes | 1 | c | >99% | — | ≧90% | 10.0 |
| Compound 31 | yes | 2 | d | >99% | — | 87% | 0.82 |
| Compound 32 | yes | 2 | d | >99% | — | ≧90% | 0.50 |
| Compound 33 | yes | 2 | d | >99% | — | ≧90% | 0.50 |
| Compound 34 | yes | 2 | d | >99% | — | ≧90% | 1.10 |

1. A molecule corresponding to formula (I) 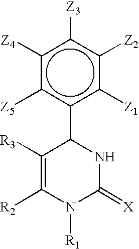
in which
X represents S;
R₁ represents a group chosen from: a hydrogen atom, a C₁-C₆ alkyl group, a C₂-C₆ alkenyl group, a C₁-C₆ haloalkyl group, a C₆-C₁₂ aralkyl group and a phenyl group optionally substituted with one or more groups chosen from: a halogen, a C₁-C₃ alkyl, a C₁-C₃ haloalkyl, a hydroxyl group and a C₁-C₃ alkoxy group;
R₂ represents a group chosen from: a C₁-C₆ alkyl group, a C₂-C₆ alkenyl group, a C₁-C₆ haloalkyl group, a C₆-C₁₂ aralkyl group and a phenyl group optionally substituted with one or more groups chosen from: a halogen, a C₁-C₃ alkyl, a C₁-C₃ haloalkyl, a hydroxyl group and a C₁-C₃ alkoxy group;
R₃ represents a group chosen from: 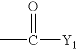
and Y₁ represents a group chosen from: a C₁-C₆ alkyl group, a C₂-C₆ alkenyl group, a C₁-C₆ haloalkyl group, a C₆-C₁₂ aralkyl group, a heteroaryl group containing up to 12 carbon atoms and from 1 to 3 heteroatoms, and a phenyl optionally substituted with one or more groups chosen from: a halogen, a C₁-C₆ alkyl, a C₁-C₆ haloalkyl, a hydroxyl group, a C₁-C₆ alkoxy group and a C₆-C₉ aralkyl group;
Z₁, Z₂, Z₄ and Z₅, which may be identical or different, being chosen from: a hydrogen atom, a halogen, a hydroxyl group, a C₁-C₆ alkyl group, a C₁-C₆ haloalkyl group, a C₁-C₆ alkoxy group and a C₁-C₁₂ acyloxy group;
Z₃ is chosen from: a hydrogen atom and a hydroxyl group;
enantiomers thereof, diastereoisomers thereof and pharmaceutically acceptable salts thereof,
with the exclusion of the compounds for which:
R₁=H or R₁=CH₃, R₂=CH₃, R₃=—CO—CH₃ and Z₁=Z₂=Z₄=Z₅=H and Z₃=H,
R₁=H, 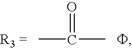
and Z₁=Z₂=Z₃=Z₄=Z₅=H, R₂=—CF₂—CF₂H, CH₃, Φ,
R₁=H, R₂=CH₃, R₃=CH₃—CO, Z₁=H, Z₂=OCH₃, Z₃=OH, Z₄=H and Z₅=H,
R₁=H, R₂=CH₃, R₃=CH₃—CO, Z₁=OCH₃ or Z₁=OCH₂CH₃, Z₂=H, Z₃=H, Z₄=H and Z₅=H.

2. The molecule as claimed in claim 1, characterized in that R₂ is selected from C₁-C₆ alkyl and C₁-C₆ haloalkyl groups.

3. The molecule as claimed in claim 2, characterized in that R₂ is chosen from: —CH₃, —CH₂—CH₃, —CH(CH₃)₂, —CH₂—CH(CH₃)₂ and —CF₃.

4. The molecule as claimed in claim 1, characterized in that (Z₁, Z₂, Z₃, Z₄, Z₅)=(H, H, H, H, Y) and Y represents a group chosen from a hydrogen atom, a hydroxyl group, a halogen atom, a C₁-C₆ alkyl group, a C₁-C₆ haloalkyl group, a C₁-C₆ alkoxy group and a C₁-C₁₂ acyloxy group.

5. The molecule as claimed in claim 4, characterized in that (Z₁, Z₃, Z₄, Z₅)=(H, H, H, H) and Z₂ represents a group chosen from a hydroxyl group and a C₁-C₆ alkoxy group.

6. The molecule as claimed in claim 4, characterized in that Y is chosen from: —OH, H, Cl, F, Br and —OCH₃.

7. The molecule as claimed in claim 1, characterized in that R₁ represents a group chosen from: a C₁-C₆ alkyl group, a C₂-C₆ alkenyl group, a C₁-C₆ haloalkyl group and a phenyl group optionally substituted with one or more groups chosen from: a halogen, a C₁-C₃ alkyl, a C₁-C₃ haloalkyl, a hydroxyl group and a C₁-C₃ alkoxy group.

8. The molecule as claimed in claim 1, characterized in that R₁ is selected from C₁-C₃ alkyls and alkenyls.

9. The molecule as claimed in claim 1, characterized in that R₁ is H.

10. The molecule as claimed in claim 1, characterized in that the group Y₁ is chosen from C₁-C₆ alkyl groups, even more preferably C₁-C₃ alkyl groups, or from phenyl groups optionally substituted with one or more substituents chosen from: —OCH₃, —F, —Cl and —Br.

11. The molecule as claimed in claim 10, characterized in that Y₁ is chosen from methyl, phenyl, meta-fluorophenyl, meta-iodophenyl, meta-chlorophenyl and meta-methoxyphenyl.

12. The molecule as claimed in claim 1, characterized in that it is chosen from the molecules below:

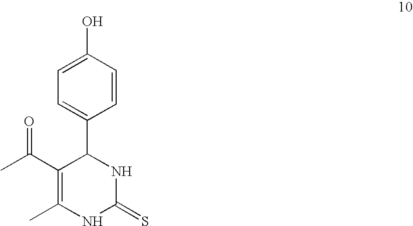

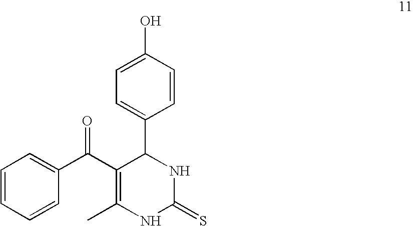

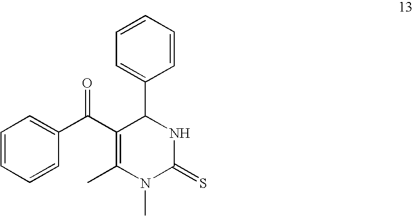

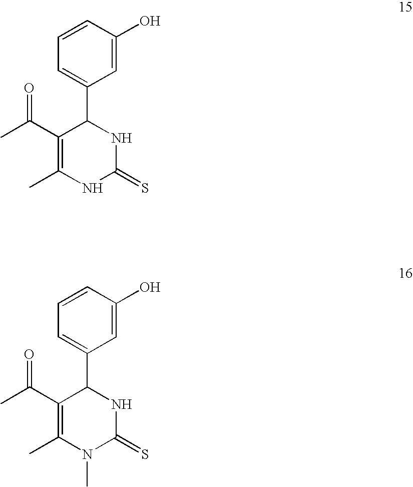

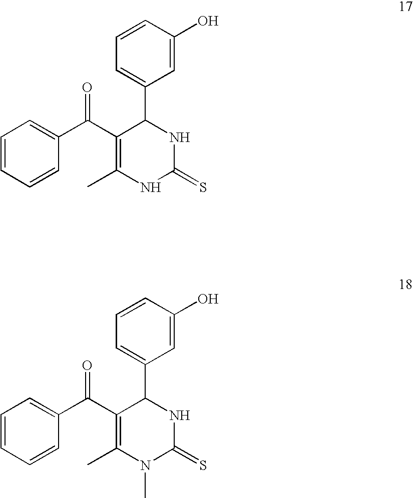

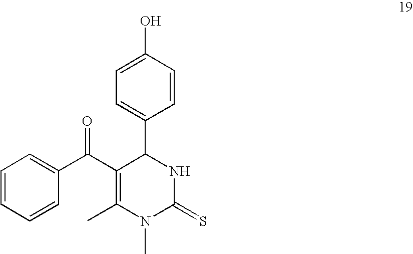

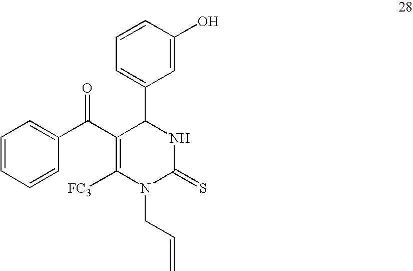

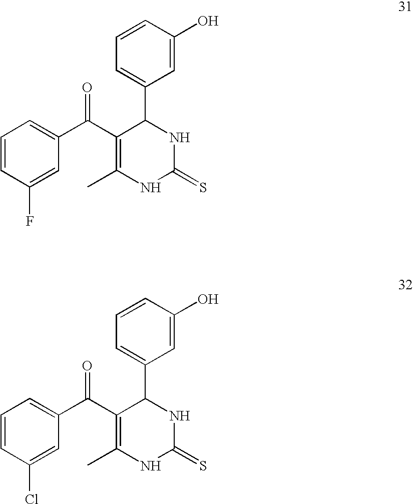

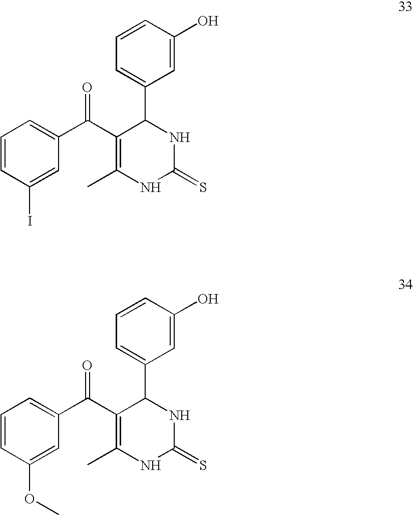

13. A process for preparing the molecules as claimed in claim 1, characterized in that the aldehyde (II), the ketone (III) and the thiourea (IV) are reacted together to give the compound (I) according to scheme 1:

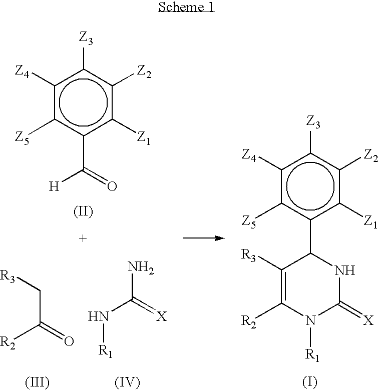

14. The process as claimed in claim 13, characterized in that the three reactants (II), (III) and (IV) are reacted in the absence of solvent, the aldehyde (II) and the ketone (III) being in an amount substantially equivalent in terms of number of moles, whereas the thiourea (IV) is present in an amount of between 1 and 2 times the amount of the aldehyde (II) or of the ketone (III), the mixture being heated to a temperature ranging from 80 to 120° C. for several hours.

15. The process as claimed in claim 13, characterized in that the reaction is carried out in the solid phase, compound (II) being attached to a solid resin via one of its functionalities Z₁, Z₂, Z₃, Z₄ or Z₅.

16. The process as claimed in claim 15, characterized in that, when Z₄=OH, compound (II) is grafted onto a resin comprising a carboxylic acid functionality by means of an esterification reaction, so as to give the functionalized resin (IIa); this resin (IIla) is then placed in the presence of the ketone (III) and the thiourea (IV) so as to give the grafted resin (Ia) from which compound (I) is detached by simple hydrolysis, according to scheme 2:

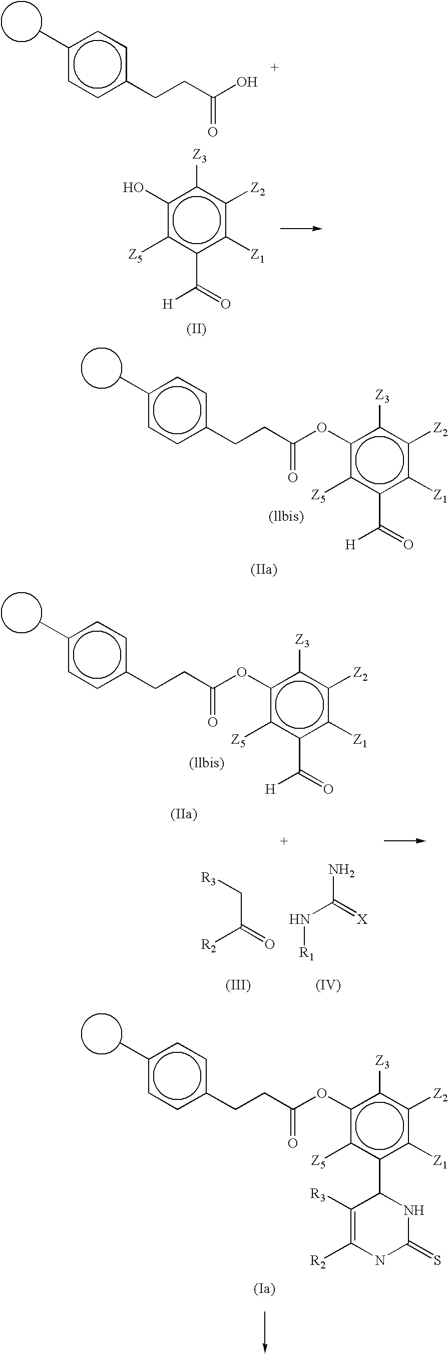

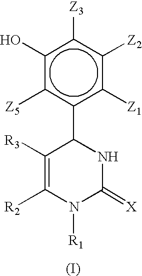

17. A medicament comprising a compound of formula (I) as claimed in claim 1, in a pharmaceutically acceptable carrier.

18. A method for the preparation of a medicament for use in the prevention and/or treatment of a proliferative disease comprising combining a compound of formula (I) as claimed in claim 1 with a pharmaceutically acceptable carrier.

19. A method for the prevention and/or treatment of a pathology selected from: cancers, autoimmune diseases, viral diseases, fungal diseases, neurodegenerative diseases and cardiovascular diseases comprising administering to a subject a compound of formula (I) as claimed in claim 1.

20. A method for the prevention and/or treatment of a cancer, in particular: lung cancer, breast cancer, pancreatic cancer, stomach cancer, ovarian cancer, esophageal cancer, thyroid cancer, prostate cancer, melanomas, lymphomas, sarcomas, carcinomas, and nervous system tumors comprising administering to a subject a compound of formula (I) as claimed in claim 1.

21. A medicament as claimed in claim 17, in combination with another medicament chosen from doxorubicin, paclitaxel, etoposide, cisplatin, tamoxifen, methotrexate and 5-fluorouracil.

22. The molecule as claimed in claim 8 wherein R₁ is selected from the groups CH₃ and CH₂—CH═CH₂.
